(19) United States
(12) Patent Application Publication
(10) Pub. No.: US 2004/0141925 A1
(43) Pub. Date: Jul. 22, 2004

(54) Novel triamcinolone compositions

(75) Inventors: H. William Bosch, Bryn Mawr, PA (US); Kevin D. Ostrander, Ringoes, NJ (US); Eugene R. Cooper, Berwyn, PA (US)

Correspondence Address: FOLEY AND LARDNER, SUITE 500, 3000 K STREET NW, WASHINGTON, DC, 20007, US

(73) Assignee: Elan Pharma International Ltd.

(21) Appl. No.: 10/697,716

(22) Filed: Oct. 31, 2003

Related U.S. Application Data
(63) Continuation-in-part of application No. 09/190,138, filed on Nov. 12, 1998.
(63) Continuation-in-part of application No. 09/337,675, filed on Jun. 22, 1999.
(63) Continuation-in-part of application No. 10/004,808, filed on Dec. 7, 2001.
Application No. 10/004,808 is a division of application No. 09/414,159, filed on Oct. 8, 1999, now Pat. No. 6,428,814.
(63) Continuation-in-part of application No. 10/345,312, filed on Jan. 16, 2003.
Application No. 10/345,312 is a continuation of application No. 09/715,117, filed on Nov. 20, 2000, now abandoned.
Application No. 10/345,312 is a continuation-in-part of application No. 10/075,443, filed on Feb. 15, 2002, now Pat. No. 6,592,903.
Application No. 10/075,443 is a continuation of application No. 09/666,539, filed on Sep. 21, 2000, now Pat. No. 6,375,986.
(63) Continuation-in-part of application No. 10/357,514, filed on Feb. 4, 2003.
(63) Continuation-in-part of application No. 10/619,539.
(60) Provisional application No. 60/353,230, filed on Feb. 4, 2002.
(60) Provisional application No. 60/396,530, filed on Jul. 16, 2002.

(51) Int. Cl. A61L 9/04; A61K 9/14

(52) U.S. Cl. 424/46; 424/489

(57) ABSTRACT

The invention is directed to nanoparticulate triamcinolone and/or triamcinolone derivative compositions. The triamcinolone or triamcinolone derivative particles of the composition have an effective average particle size of less than about 2 microns.

### CROSS-REFERENCE TO RELATED PATENT APPLICATIONS

This application is a continuation in part of U.S. patent application Ser. No. 10/004,808, filed on Dec. 7, 2001 (pending), which is a divisional of U.S. patent application Ser. No. 09/414,159, filed on Oct. 8, 1999, now U.S. Pat. No. 6,428,814. In addition, this application is a continuation-in-part of: (a) U.S. patent application Ser. No. 09/190,138, filed on Nov. 12, 1998 (pending); (b) U.S. patent application Ser. No. 10/619,539, filed on Jul. 16, 2003 (pending), which claims priority of U.S. patent application Ser. No. 60/396,530, filed on Jul. 16, 2002; (c) U.S. patent application Ser. No. 09/337,675, filed on Jun. 22, 1999 (pending); (d) U.S. patent application Ser. No. 10/357,514, filed on Feb. 4, 2003 (pending), which claims priority of U.S. patent application Ser. No. 60/353,230, filed on Feb. 4, 2002; and (e) U.S. patent application Ser. No. 10/345,312, filed on Jan. 16, 2003 (pending), which is a continuation of U.S. patent application Ser. No. 09/715,117, filed on Nov. 20, 2000 (now abandoned), and a continuation-in-part of U.S. patent application Ser. No. 10/075,443, filed on Feb. 15, 2002, now U.S. Pat. No. 6,592,903, which is a continuation of U.S. patent application Ser. No. 09/666,539, filed on Sep. 21, 2000, now U.S. Pat. No. 6,375,986. The prior disclosures are specifically incorporated by reference.

### FIELD OF THE INVENTION

The present invention relates to novel compositions of triamcinolone and triamcinolone derivatives, comprising particles of triamcinolone or derivatives thereof having an effective average particle size of less than about 2000 nm and at least one surface stabilizer that is preferably adsorbed to or associated with the surface of the triamcinolone particles.

### BACKGROUND OF THE INVENTION

I. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having associated with the surface thereof a non-crosslinked surface stabilizer. The '684 patent does not describe nanoparticulate triamcinolone or triamcinolone derivative compositions.

Methods of making nanoparticulate active agent compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.” These patents do not describe methods of making nanoparticulate triamcinolone or triamcinolone derivatives.

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate,” U.S. Pat. No. 6,428,814 for “Bioadhesive nanoparticulate compositions having cationic surface stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract,” and U.S. Pat. No. 6,592,903 for “Nanoparticulate Dispersions Comprising a Synergistic Combination of a Polymeric Surface Stabilizer and Dioctyl Sodium Sulfosuccinate,” all of which are specifically incorporated by reference. In addition, U.S. patent application Ser. No. 20020012675 A1, published on Jan. 31, 2002, for “Controlled Release Nanoparticulate Compositions,” and WO 02/098565 for “System and Method for Milling Materials,” describe nanoparticulate active agent compositions, and are specifically incorporated by reference. None of these references describe nanoparticulate compositions of triamcinolone or triamcinolone derivatives.

Amorphous small particle compositions are described, for example, in U.S. Pat. Nos. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” These references do not describe nanoparticulate triamcinolone and triamcinolone derivatives.

II. Background Regarding Triamcinolone and Triamcinolone Derivatives

Triamcinolone and derivatives thereof (“triamcinolone derivatives”) are corticosteroids of the glucocorticoid family. Glucocorticoids have anti-inflammatory properties and are useful in the treatment of inflammation (swelling, heat, redness, and pain). Glucocorticoids are used to treat certain forms of arthritis, skin, blood, kidney, eye, thyroid, and intestinal disorders (e.g., colitis, irritable bowel disorder, and Crohn's disease), allergies, and asthma. Glucocorticoids are also is administered with other drugs to prevent rejection of transplanted organs and to treat certain types of cancer.

Triamcinolone and triamcinolone derivatives, such as triamcinolone acetonide, triamcinolone diacetate, triamcinolone hexacetonide, and triamcinolone benetonide, are all derived from the naturally occurring parent compound prednisone. Predisone and prednisolone are members of the glucocorticoid class of hormones. Naturally occurring corticosteroids have varying glucocorticoid and mineralocorticoid activities. Synthetic corticosteroids are modifications of the parent molecule typically having improved anti-inflammatory activity and reduced mineralocorticoid activity. Additionally, the modifications to the parent corticosteroid alter the water solubility of the drug, which is thought to be associated with the duration of action. For example, triamcinolone and triamcinolone acetonide are two-fold and 8-fold more active, respectively, than prednisone in animal models of inflammation. See The Physicians' Desk Reference, 56th Ed., pp. 728 (Thompson P D R, Montvale N.J., 2002).

In general, corticosteroids with succinate and phosphate esters are water soluble, have a short duration of action, and are good for parenteral use. Corticosteroids with acetate and acetonide esters are more lipid-soluble, have a longer duration of action, and are useful for in situ administration (i.e., intra-artricular or intrabursal administaion). Thus, suitable applications and administrative routes are governed by the particular triamcinolone compound with its inherent solubility and pharmacokinetic properties.

A. Chemical Properties of Triamcinolone and Triamcinolone Derivatives

1. Triamcinolone

Triamcinolone, also known as (11 [symbol] ,16 [symbol] )-9-fluoro-11,17,18,21-dihydroxy-pregna-1,4-diene-3,20-dione, has the following chemical structure:

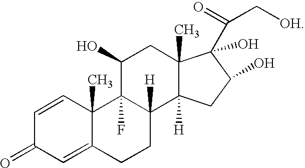

Triamcinolone has an empirical formula of C₂₁H₂₇FO₆ and a molecular weight of 394.43. See The Merck Index, 13th Ed., pp. 1711-2 (Merck & Co. 2001). Triamcinolone is slightly soluble in water, very slightly soluble in chloroform and ether, 1 part into 40 parts soluble in alcohol, and slightly soluble in methyl alcohol. Chem. Abstracts Registration No. 124-94-7.

A comprehensive description of triamcinolone is disclosed in, e.g., Florey, Anal. Profiles Drug Subs. 1:367-96, 423-442 (1972) and Sieh, Ibid. 11:593-614, 651-661 (1982). Methods of preparing triamcinolone are disclosed in, e.g., U.S. Pat. Nos. 2,789,118 and 3,021,347.

2. Triamcinolone Acetonide

Triamcinolone acetonide, also known as (11 [symbol] , 16 [symbol] )-9-fluoro-11,21-dihydroxy-16,17-[1-methylethylidenebis(oxy)]-pregna-1,4-diene-3,20-dione, has the following chemical structure:

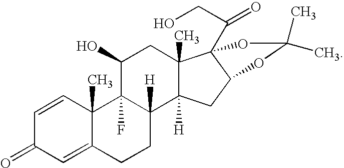

Triamcinolone acetonide has an empirical formula of C₂₄H₃₁FO₆ and a molecular weight of 434.5. See The Physicians' Desk Reference, 56th Ed., pp. 728 (Thompson P D R, Montvale N.J., 2002) and The Merck Index at 1712. Triamcinolone acetonide is sparingly soluble in methane, acetone, and ethyl acetate. The Merck Index at 1712.

Triamcinolone acetonide is described in, e.g., Florey, Anal. Profiles Drug Subs., 1:397 (1972) and Sieh, Ibid., 11:615 (1982). Methods of preparing triamcinolone acetonide are disclosed in, e.g., Fried et al., J. Am. Chem Soc., 80:2238 (1958), Bernstein et al., Ibid., 81:1689 (1959), and U.S. Pat. Nos. 2,990,401 and 3,035,050. Clinical trials of triamcinolone acetonide in chronic asthma are disclosed in Bernstein et al., Chest, 81:20 (1982).

3. Triamcinolone Diacetate

Triamcinolone diacetate, also known as (11 [symbol] ,16 [symbol] )-16,21-bis(acetyloxy)-9-fluoro-11,17-dihydroxypregna-1,4-diene-3,20-dione, has the following chemical formula:

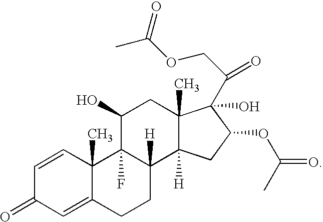

Triamcinolone diacetate has an empirical formula of C₂₅H₃₁FO₈ and a molecular weight of 478.51. See The Physicians' Desk Reference at 1388 (Thompson P D R, Montvale N.J., 2002) and The Merck Index at 1712. Triamcinolone diacetate is sparingly soluble in water. The Merck Index at 1712.

4. Triamcinolone Hexacetonide

Triamcinolone hexacetonide, also known as (11 [symbol] , 16 [symbol] )-21-(3,3dimethyl-1-oxobutoxy)-9-fluoro-11-hydroxy-16,17-[(1-methylethylidene)bis(oxy)]-pregna-1,4-diene-3,20-dione, has the following formula:

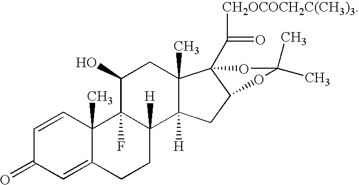

Triamcinolone hexacetonide has an empirical formula of C₃₀H₄₁FNO₇ and a molecular weight of 532.64. See The Physician's Desk Reference at 1390 and The Merck Index at 1712. Triamcinolone hexacetonide has the following solubilities in g/100 ml solvent at 25° C.: >5 in chloroform and dimethylacetamide, 0.77 in acetate, 0.59 in methanol, 0.5 in diethyl carbonate, 0.42 in glycerin, 0.13 in propylene glycol, 0.03 in absolute alcohol, and 0.0002-0.0004 in water. Id.

A description of triamcinolone hexacetonide is provided in, e.g., Zbinovsky and Chrekian, Anal. Profiles Drug Subs., 6:579-95 (1977). Methods of preparing triamcinolone hexacetonide are disclosed in, e.g., U.S. Pat. No. 3,457,348. The anti-inflammatory properties of triamcinolone hexacetonide in rabbits are disclosed in Hunneyball, Agents Actions, 11:490 (1981) and early clinical studies are disclosed in Bilka, Minnesota Med., 50:483 (1967) and Layman and Peterson, Id. at 669.

5. Triamcinolone Benetonide

Triamcinolone benetonide, also known as (110, 160)-21-[3-(benzoylamino)-2-methyl-1-oxopropoxy]-9-fluoro-11-hydroxy-16,17-[(1-methylethylidene)bis(oxy)]-pregna-1,4-diene-3,20-dione, has the following chemical structure:

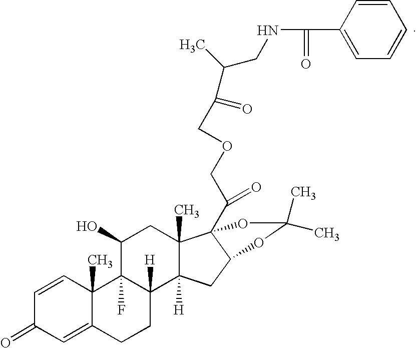

Triamcinolone benetonide has an empirical formula of C₃₅H₄₂FNO₈ and a molecular weight of 623.71. See The Merck Index at 1712. Triamcinolone benetonide is soluble in methanol, acetone, ethanol, dioxane, pyridine, DMF, and chloroform, but is insoluble in water. Id.

Methods of preparing triamcinolone benetonide are disclosed in, e.g., U.S. Pat. Nos. 3,749,712 and 3,035,050. Clinical studies of triamcinolone benetonide are disclosed in Tazelaar, J. Int. Med. Res., 5:338 (1981). Down et al, Toxicol Letters, 1:95 (1977), describes percutaneous absorption of triamcinolone benetonide in rats and rabbits.

B. Pharmaceutical Applications of Triamcinolone and Triamcinolone Derivatives

Triamcinolone and triamcinolone derivatives are indicated for the treatment of conditions where a powerful steroidal anti-inflammatory agent is required. Such conditions include, but are not limited to, asthma, contact dermatitis, atopic dermatitis, seasonal or perennial allergic rhinitis, oral inflammatory, lesions and ulcers, osteoarthritis, acute nonspecific and posttraumatic osteoarthritis, rheumatoid arthritis, bursitis, epicondylitis, keloids, and skin disorders including psoriasis, eczema, and general dermatitis. Additional conditions where triamcinolone and triamcinolone derivatives are useful include, but are not limited to, endocrine disorders, lupus, acute rheumatic carditis, herpes zoster ophthalmicus, intestinal disorders (e.g., irritable bowel disorder, colitis, ulcerative colitis, gastroenteritis, and Crohn's disease), hemolytic anemia, and neoplastic diseases such as leukemias and lymphoma.

Due to the broad range of uses, triamcinolone and triamcinolone derivatives are currently available in a variety of formulations suitable for various administrative routes including oral/pulmonary inhalation, nasal inhalation, topical application, and injectable formulations. Triamcinolone and triamcinolone derivatives are formulated as an oral/pulmonary and nasal aerosol, aqueous nasal spray, dental paste, injectable forms (intra-articular, intrabursal administration, intradermal, intramuscular), and topical formulations (aerosol spray, cream, ointment, and lotion). Triamcinolone and triamcinolone derivatives are also available mixed with other drugs, such as anti-fungal agents.

For example, triamcinolone is available under the brand name KENALOG® (Bristol-Myers Squibb Pharmaceuticals Ltd., Hounslow, England) in topical formulations (e.g. aerosol, lotion, cream, ointment, and dental paste) indicated for the treatment of skin irritation including irritation caused by allergic reactions, psoriasis, eczema, and dermatitis. Triamcinolone is also available in injectable formulations marketed under the brand names KENALOG®, KENALOG-10®, KENALOG-40®, KENAJECT®.

Triamcinolone acetonide is currently marketed for oral/pulmonary inhalation under the brand name AZMACORT® (Aventis Pharmaceuticals, Inc., Bridgewater, N.J., USA). AZMACORT® is indicated for the treatment of bronchial asthma and is supplied as an inhalation aerosol containing a 1% w/w microcrystalline suspension of triamcinolone acetonide in the propellant dichlorodifluoromethane and dehydrated alcohol. See The Physician's Desk Reference at 728.

Triamcinolone acetonide is also marketed under the brand names NASACORT® and NASACORT® AQ (Aventis Pharmaceuticals, Inc.) in the form of nasal inhalants for the treatment of the nasal symptoms of seasonal and perennial allergic rhinitis. See The Physician's Desk Reference at 750-3. When administered intranasally triamcinolone acetonide has a direct anti-inflammatory effect on the nasal mucosa. NASACORT® is provided as a 0.7% microcrystalline suspension of triamcinolone acetonide in dichlorodifluoromethane and dehydrated alcohol. NASACORT® AQ is an aqueous, thixotropic suspension of microcrystalline triamcinolone acetonide. The aqueous pharmaceutical composition of NASACORT® AQ is disclosed in U.S. Pat. Nos. 5,976,573 and 6,143,329.

Triamcinolone acetonide is also available in cream, lotion, and ointment formulations indicated for the treatment of itching, redness, dryness, crusting, scaling, inflammation, and discomfort of various skin conditions. For example, ARISTOCORT A® is available as a 0.025%, 0.1% or 0.5% triamcinolone acetonide in AQUATAIN® hydrophilic base (Fujisawa Pharmaceutical Co., Ltd. Japan) or ointment (ARISTOCORT A® 0.1% ointment with propylene glycol). Additional products containing triamcinolone acetonide include nystatin and triamcinolone acetonide cream and ointment (Taro, Hawthorne, N.Y., USA), triamcinolone acetonide cream, lotion, and ointment (Watson Laboratories, Corona, Calif., USA), and triamcinolone acetonide dental paste (Taro).

Triamcinolone diacetate is marketed under the brand names ARISTOCORT® and ARISTOCORT® Forte (Fujisawa Healthcare, Deerfield, Ill., USA). ARISTOCORT® is available as a suspension of 25 mg/mL micronized triamcinolone diacetate. ARISTOCORT® is indicated for intralesional injection only. The Physicians' Desk Reference at 1388. ARISTOCORT® Forte is supplied as a suspension of 40 mg/mL triamcinolone diacetate suitable for parenteral administration. ARISTOCORT® Forte can be administered via intramuscular, intra-articular, or intra-synovial routes but is not for intravenous administration.

Triamcinolone hexacetonide is formulated for parenteral administration via intra-articular, intralesional or sublesional routes. Triamcinolone hexacetonide is marketed under the brand name ARISTOSPAN® and is available as an aqueous suspension of 5 mg/mL or 20 mg/mL micronized triamcinolone hexacetonide.

Triamcinolone benetonide is available as a topical anti-inflammatory in a 0.075% cream.

The pharmacokinetic properties of triamcinolone acetonide are known. See The Physician's Desk Reference at 728, 750 and 752. Triamcinolone acetonide, administered intravenously as the phosphate ester, has a plasma half-life (T_{1/2}) of 88 minutes. The plasma half-life of glucocorticoids generally does not correlate well with the biologic half-life.

Following a single oral dose of 800 mcg ¹⁴C-labeled triamcinolone acetonide, the maximum plasma concentration was achieved in 1.5 to 2 hours (Tₘₐₓ). Plasma protein binding of triamcinolone acetonide appears to be relatively low and consistent over a wide plasma concentration range. The overall mean percent fraction of triamcinolone acetonide by plasma proteins was approximately 68%. No triamcinolone acetonide was detected in the plasma after 24 hours and greater than 90% of the oral [¹⁴C]-radioactive dose was recovered within 5 days after administration in 5 out of the 6 test subjects. Of the recovered [¹⁴C]-radioactivity, approximately 40% and 60% was found in the urine and feces, respectively.

Intranasal administration of 440 mcg/day dose of NASACORT® resulted in a Cₘₐₓ of <1 ng/mL and a Tₘₐₓ of 3.4 hours (ranging from 0.5 to 8.0 hours). The apparent T_{1/2} was 4.0 hours±3 hours.

Single dose intranasal administration of 220 mcg of NASACORT® AQ Nasal Spray produced a Cₘₐₓ of approximately 0.5 ng/mL±0.5 ng/mL and a Tₘₐₓ of approximately 1.5 hours. The average T_{1/2} of triamcinolone acetonide was 3.1 hours, the concentration was less than 0.06 ng/mL at 12 hours and below the assay detection limit at 24 hours. The mean AUC values ranged from 1.4 ng-hr/mL to 4.7 ng-hr/mL over the range of 110 mcg to 440 mcg. The Cₘₐₓ was linear over the range of 110 mcg or 220 mcg when administered intranasally. Following multiple doses in pediatric patients receiving 440 mcg/day, plasma drug concentrations pharmacokinetic parameters were similar to those values observed in adult patients.

C. Adverse Properties of Triamcinolone and Triamcinolone Derivatives

Corticosteroid administration can result in significant side effects. The most frequent side effects of inhaled triamcinolone and triamcinolone derivates are mild coughing or wheezing and occasional oral candidiasis infection. Other nasopharyngeal side effects include dry mucous membranes, sinus congestion, throat discomfort, sneezing, and epistaxis. Side effects apparent with topical administration of triamcinolone and triamcinolone derivates to the skin are burning, itching, irritation, dryness, folliculitis, hypertrichosis, acne, hypopigmentation, perioral dermatitis, allergic contact dermatitis, maceration of the skin, and secondary infection.

Higher doses of inhaled glucocorticoids are believed to decrease bone formation or increase bone reabsorption, resulting in weak bones and increased instances of bone fractures. Corticosteroids, in general, are known to cause decreased resistance to localized infections and to inhibit wound healing.

Systemic absorption of topical corticosteroids can result in reversible hypothalamic-pituitary-adrenal (HPA) axis suppression, Cushing's syndrome-like symptoms, and hyperglycemia. Although complete recovery of HPA axis function is typically prompt upon discontinuing the drug, signs and symptoms of steroid withdrawal can occur, requiring supplemental systemic corticosteroids.

Children are especially susceptible to systemic toxicity caused by corticosteroids. In addition to the symptoms experienced by adults, intracranial hypertension, retardation of growth and development, delayed weight gain, low plasma cortisol levels, and absence of response to ACTH stimulation has been reported in children receiving corticosteroids. Accordingly, administration of corticosteroids to children should be limited.

Although topical (including inhaled) administration of corticosteroids, such as triamcinolone and triamcinolone derivatives, minimizes the side-effects as compared to systemic administration, the active compounds are still absorbed into the circulation where they are systemically active.

There is a need in the art for triamcinolone acetonide compositions which can decrease frequency of dosing, improve clinical efficacy, and potentially reduce side effects. The present invention satisfies these needs.

### SUMMARY OF THE INVENTION

The present invention relates to nanoparticulate compositions comprising triamcinolone, triamcinolone derivatives, or a mixture thereof. The compositions comprise triamcinolone and/or triamcinolone derivatives and at least one surface stabilizer, which is preferably adsorbed on or associated with the surface of the triamcinolone or triamcinolone derivative particles. The nanoparticulate triamcinolone and triamcinolone derivative particles have an effective average particle size of less than about 2 microns.

Another aspect of the invention is directed to pharmaceutical compositions comprising the nanoparticulate triamcinolone or triamcinolone derivative compositions of the invention. The pharmaceutical compositions preferably comprise triamcinolone and/or a triamcinolone derivative, at least one surface stabilizer, and at least one pharmaceutically acceptable carrier, as well as any desired excipients. Advantages and properties of the compositions of the invention are described herein.

The invention further discloses a method of making nanoparticulate triamcinolone and triamcinolone derivative compositions. Such a method comprises contacting triamcinolone and/or a triamcinolone derivative and at least one surface stabilizer for a time and under conditions sufficient to provide a nanoparticulate triamcinolone and/or triamcinolone derivative composition. The one or more surface stabilizers can be contacted with triamcinolone and/or a triamcinolone derivative either before, preferably during, or after size reduction of the triamcinolone and/or triamcinolone derivative.

The present invention is also directed to methods of treatment using the nanoparticulate triamcinolone and triamcinolone derivative compositions of the invention for treatment of conditions or disorders where a glucocorticoid composition is useful. Such conditions or disorders include, but are not limited to, arthritis, skin disorders, blood disorders, kidney disorders, eye disorders, thyroid, intestinal disorders, cancer, allergic reactions, asthma, contact dermatitis, atopic dermatitis, seasonal allergic rhinitis, perennial allergic rhinitis, oral inflammatory, oral lesions, oral ulcers, osteoarthritis, acute nonspecific osteoarthritis, posttraumatic osteoarthritis, rheumatoid arthritis, bursitis, epicondylitis, psoriasis, eczema, general dermatitis, endocrine disorders, lupus, herpes zoster ophthalmicus, ulcerative colitis, irritable bowel disorder, Crohn's disease, gastroenteritis, hemolytic anemia, leukemia, and lymphoma.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to nanoparticulate compositions comprising triamcinolone and/or at least one triamcinolone derivative. The compositions comprise triamcinolone and/or a triamcinolone derivative and at least one surface stabilizer that is preferably adsorbed on or associated with the surface of the drug. The nanoparticulate particles of triamcinolone and/or a triamcinolone derivative have an effective average particle size of less than about 2 microns.

As taught in the '684 patent, not every combination of surface stabilizer and active agent will result in a stable nanoparticulate active agent composition. It was surprisingly discovered that stable nanoparticulate formulations of triamcinolone and triamcinolone derivatives can be made.

The current formulations of triamcinolone and triamcinolone derivatives suffer from the following problems: (1) the extremely poor solubility of the drugs results in low bioavailability; (2) for some uses, dosing must be repeated several times each day; and (3) a wide variety of side effects are associated with the current dosage forms of the drug.

The present invention overcomes problems encountered with the prior art formulations of triamcinolone and triamcinolone derivatives. Specifically, the nanoparticulate triamcinolone and triamcinolone derivative compositions of the invention may offer the following advantages as compared to prior art compositions of conventional non-nanoparticulate triamcinolone and non-nanoparticulate triamcinolone derivatives: (1) faster onset of action; (2) a potential decrease in the frequency of dosing; (3) smaller doses of triamcinolone and triamcinolone derivatives required to obtain the same pharmacological effect; (4) increased bioavailability; (5) an increased rate of dissolution; (6) improved performance characteristics for oral, intravenous, subcutaneous, or intramuscular injection, such as higher dose loading and smaller tablet or liquid dose volumes; (7) improved pharmacokinetic profiles, such as improved Tₘₐₓ, Cₘₐₓ, and AUC profiles; (8) substantially similar or bioequivalent pharmacokinetic profiles of the nanoparticulate triamcinolone and triamcinolone derivative compositions when administered in the fed versus the fasted state; (9) bioadhesive triamcinolone and triamcinolone derivative formulations, which can coat the gut, mucous membranes, or the desired site of application and be retained for a period of time, thereby increasing the efficacy of the drug as well as eliminating or decreasing the frequency of dosing; (10) high redispersibility of the nanoparticulate triamcinolone and triamcinolone derivative particles present in the compositions of the invention following administration; (11) the nanoparticulate triamcinolone and triamcinolone derivative compositions can be formulated in a dried form which readily redisperses; (12) low viscosity liquid nanoparticulate triamcinolone and triamcinolone derivative dosage forms can be made; (13) liquid nanoparticulate triamcinolone and triamcinolone derivative compositions having a low viscosity result in better subject compliance due to the perception of a lighter formulation which is easier to consume and digest; (14) liquid nanoparticulate triamcinolone and triamcinolone derivative compositions having a low viscosity result in greater ease of dispensing because one can use a cup or a syringe; (15) the nanoparticulate triamcinolone and triamcinolone derivative compositions can be used in conjunction with other active agents; (16) the nanoparticulate triamcinolone and triamcinolone derivative compositions can be sterile filtered; (17) the nanoparticulate triamcinolone and triamcinolone derivative compositions are suitable for parenteral administration; and (18) the nanoparticulate triamcinolone and triamcinolone derivative compositions do not require organic solvents or pH extremes.

Preferred dosage forms of the invention are aerosol (pulmonary and nasal), liquid suspension, and oral tablet formulations, although any pharmaceutically acceptable dosage form can be utilized. Other preferred dosage forms include topical cream, lotion and ointment formulations.

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

“Conventional” or “non-nanoparticulate active agent” shall mean an active agent which is solubilized or which has an effective average particle size of greater than about 2 microns. Nanoparticulate active agents as defined herein have an effective average particle size of less than about 2 microns.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” as used herein refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Poorly water soluble drugs” as used herein means those having a solubility of less than about 30 mg/ml, preferably less than about 20 mg/ml, preferably less than about 10 mg/ml, or preferably less than about 1 mg/ml. Such drugs tend to be eliminated from the gastrointestinal tract before being absorbed into the circulation.

As used herein with reference to stable drug particles, ‘stable’ includes, but is not limited to, one or more of the following parameters: (1) that the triamcinolone and triamcinolone derivative particles do not appreciably flocculate or agglomerate due to interparticle attractive forces, or otherwise significantly increase in particle size over time; (2) that the physical structure of the triamcinolone and triamcinolone derivative particles are not altered over time, such as by conversion from an amorphous phase to crystalline phase; (3) that the triamcinolone and triamcinolone derivative particles are chemically stable; and/or (4) where the triamcinolone and triamcinolone derivative has not been subject to a heating step at or above the melting point of the triamcinolone and triamcinolone derivative in the preparation of the nanoparticles of the invention.

‘Therapeutically effective amount’ as used herein with respect to a drug dosage, shall mean that dosage that provides the specific pharmacological response for which the drug is administered in a significant number of subjects in need of such treatment. It is emphasized that ‘therapeutically effective amount,’ administered to a particular subject in a particular instance will not always be effective in treating the diseases described herein, even though such dosage is deemed a ‘therapeutically effective amount’ by those skilled in the art. It is to be further understood that drug dosages are, in particular instances, measured as oral dosages, or with reference to drug levels as measured in blood.

I. Preferred Characteristics of the Nanoparticulate Triamcinolone and Triamcinolone Derivative Compositions of the Invention

A. Increased Bioavailability, Frequency of Dosing and Dosage Quantity

The nanoparticulate triamcinolone and triamcinolone derivative compositions of the invention may preferably exhibit increased bioavailability and require smaller doses as compared to prior non-nanoparticulate triamcinolone acetonide compositions administered at the same dose.

Any drug, including triamcinolone and triamcinolone derivatives, can have adverse side effects. Thus, lower doses of triamcinolone and triamcinolone derivatives which can achieve the same or better therapeutic effects as those observed with larger doses of non-nanoparticulate triamcinolone and triamcinolone derivative compositions are desired. Such lower doses may be realized with the nanoparticulate triamcinolone and triamcinolone derivative compositions of the invention because the nanoparticulate triamcinolone and triamcinolone derivative compositions may exhibit greater bioavailability as compared to non-nanoparticulate triamcinolone and triamcinolone derivative formulations, which means that smaller doses of triamcinolone and triamcinolone derivative are likely required to obtain the desired therapeutic effect.

In addition, conventional non-nanoparticulate triamcinolone and triamcinolone derivative compositions typically require multiple doses per day and often require multiple administrations per dose.

For adults, the typical recommended dosage of AZMACORT is 200 mcg 3-4 times daily or 400 mcg twice daily. For children 6 to 12 years of age, the typical dosage is 100 to 200 mcg 3-4 times daily or 200-400 mcg twice daily.

The recommended dosage of NASACORT® and NASACORT® AQ is two sprays/nostrile/day (220 mcg) which can be increased for greater efficacy to four sprays/nostrile/day (440 mcg) depending on individual patient response.

Injectable conventional formulations can also require multiple administrations or doses per day. The recommended parenteral dosage of KENALOG®-10 and 40 ranges from about ⅓ to ½ of the oral dose given every 12 hours.

For intrabursal administration, the manufacturer recommends single injections into several joints for multiple locus involvement, up to 60 mg. For intradermal administration, the initial dose of a triamcinolone acetonide composition will vary depending upon the disease being treated but should be limited to 1.0 mg (0.1 ml) per injection site because larger volumes can produce cutaneous atrophy. The manufacturer recommends injection into multiple sites (separated by one centimeter or more) to allow for administration of a sufficient amount of drug without the complications associated with large volumes.

For intra-articular or intra-bursal administration and for injection into tendon sheaths, the initial recommended dose varies from 2.5 to 5 mg triamcinolone for smaller joints and from 5 to 15 mg for larger joints. A single local injection may sufficient, but several injections may be required for adequate relief of symptoms.

The conventional topical formulations of triamcinolone and triamcinolone derivatives (e.g., lotions, creams, ointments, and dental paste) require two to four applications daily.

In contrast, the triamcinolone and triamcinolone derivative compositions of the invention may be administered less frequently and at lower doses in forms such as liquid dispersions, powders, sprays, solid re-dispersable dosage forms, ointments, creams, etc. Exemplary types of formulations useful in the present invention include, but are not limited to, liquid dispersions, sachets, lozenges, oral suspensions, gels, aerosols (pulmonary and nasal), ointments, creams, solid dose forms, tablets, capsules, and powders etc. of nanoparticulate triamcinolone and triamcinolone derivatives. Lower dosages can be used because the small particle size of the particles ensure greater absorption, and in the case of bioadhesive nanoparticulate triamcinolone and triamcinolone derivative compositions, the triamcinolone and triamcinolone derivative is retained at the desired site of application for a longer period of time as compared to conventional triamcinolone and triamcinolone derivative dosage forms.

In one embodiment of the invention, the therapeutically effective amount of the nanoparticulate triamcinolone and triamcinolone derivative compositions is ⅙, ⅕, ¼, ⅓, or ½ of the therapeutically effective amount of a non-nanoparticulate triamcinolone and triamcinolone derivative composition.

Such lower doses are preferred as they may decrease or eliminate adverse effects of the drug. In addition, such lower doses decrease the cost of the dosage form and may increase patient compliance.

B. Pharmacokinetic Profiles of the Nanoparticulate Triamcinolone and Triamcinolone Derivative Compositions of the Invention

The invention also preferably provides triamcinolone and triamcinolone derivative compositions having a desirable pharmacokinetic profile when administered to mammalian subjects.

The desirable pharmacokinetic profile of the triamcinolone and triamcinolone derivative compositions preferably includes, but is not limited to: (1) a Tₘₐₓ for triamcinolone or a triamcinolone derivative composition, when assayed in the plasma of a mammalian subject following administration, that is preferably less than the Tₘₐₓ for a non-nanoparticulate triamcinolone or triamcinolone derivative composition, administered at the same dosage; (2) a Cₘₐₓ for triamcinolone or a triamcinolone derivative composition, when assayed in the plasma of a mammalian subject following administration, that is preferably greater than the Cₘₐₓ for a non-nanoparticulate triamcinolone or triamcinolone derivative composition, administered at the same dosage; and/or (3) an AUC for triamcinolone or a triamcinolone derivative composition, when assayed in the plasma of a mammalian subject following administration, that is preferably greater than the AUC for a non-nanoparticulate triamcinolone or triamcinolone derivative composition, administered at the same dosage.

The desirable pharmacokinetic profile, as used herein, is the pharmacokinetic profile measured after the initial dose of triamcinolone or triamcinolone derivative. The compositions can be formulated in any way as described below and as known to those of skill in the art.

The use of conventional non-nanoparticulate formulations of triamcinolone and triamcinolone derivatives for treatment of asthma, allergic rhinitis, skin disorders and other inflammation-related conditions is not ideal due to delayed onset of action. In contrast, the nanoparticulate triamcinolone and triamcinolone derivative compositions of the invention exhibit faster therapeutic effects.

Preferred triamcinolone and triamcinolone derivative compositions of the invention exhibit, in comparative pharmacokinetic testing with a non-nanoparticulate triamcinolone or triamcinolone derivative composition, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate triamcinolone or triamcinolone derivative composition.

Preferred triamcinolone and triamcinolone derivative compositions of the invention exhibit, in comparative pharmacokinetic testing with a non-nanoparticulate triamcinolone or triamcinolone derivative composition, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate triamcinolone or triamcinolone derivative composition.

Preferred triamcinolone and triamcinolone derivative compositions of the invention exhibit, in comparative pharmacokinetic testing with a non-nanoparticulate triamcinolone or triamcinolone derivative composition, administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate triamcinolone or triamcinolone derivative composition.

Any formulation giving the desired pharmacokinetic profile is suitable for administration according to the present methods. Exemplary types of formulations giving such profiles are liquid dispersions, gels, aerosols, ointments, creams, solid dose forms, etc. of nanoparticulate triamcinolone and triamcinolone derivatives.

C. The Pharmacokinetic Profiles of the Nanoparticulate Triamcinolone and Triamcinolone Derivative Compositions of the Invention are Preferably not Substantially Affected by the Fed or Fasted State of the Subject Ingesting the Compositions

The invention encompasses nanoparticulate triamcinolone and triamcinolone derivative compositions wherein preferably the pharmacokinetic profile of the triamcinolone or triamcinolone derivative is not substantially affected by the fed or fasted state of a subject ingesting the composition. This means that there is no substantial difference in the quantity of triamcinolone or triamcinolone derivative absorbed or the rate of triamcinolone or triamcinolone derivative absorption when the nanoparticulate triamcinolone and triamcinolone derivative compositions are administered in the fed versus the fasted state. Thus, the nanoparticulate triamcinolone and triamcinolone derivative compositions of the invention can substantially eliminate the effect of food on the pharmacokinetics of triamcinolone and triamcinolone derivative.

In another embodiment of the invention, the pharmacokinetic profile of the triamcinolone and triamcinolone derivative compositions of the invention, when administered to a mammal in a fasted state, is bioequivalent to the pharmacokinetic profile of the same triamcinolone or triamcinolone derivative composition administered at the same dosage, when administered to a mammal in a fed state. “Bioequivalency” is preferably established by a 90% Confidence Interval (CI) of between 0.80 and 1.25 for both Cₘₐₓ and AUC under U.S. Food and Drug Administration (USFDA) regulatory guidelines, or a 90% CI for AUC of between 0.80 to 1.25 and a 90% CI for Cₘₐₓ of between 0.70 to 1.43 under the European Medicines Evaluation Agency (EMEA) regulatory guidelines (Tₘₐₓ is not relevant for bioequivalency determinations under USFDA and EMEA regulatory guidelines).

Preferably the difference in AUC (e.g., absorption) of the nanoparticulate triamcinolone or triamcinolone derivative compositions of the invention, when administered in the fed versus the fasted state, is less than about 100%, less than about 90%, less than about 80%, less than about 70%, less than about 60%, less than about 50%, less than about 40%, less than about 35%, less than about 30%, less than about 25%, less than about 20%, less than about 15%, less than about 10%, less than about 5%, or less than about 3%.

In addition, preferably the difference in Cₘₐₓ of the nanoparticulate triamcinolone and triamcinolone derivative compositions of the invention, when administered in the fed versus the fasted state, is less than about 100%, less than about 90%, less than about 80%, less than about 70%, less than about 60%, less than about 50%, less than about 40%, less than about 35%, less than about 30%, less than about 25%, less than about 20%, less than about 15%, less than about 10%, less than about 5%, or less than about 3%.

Finally, preferably the difference in the Tₘₐₓ of the nanoparticulate triamcinolone and triamcinolone derivative compositions of the invention, when administered in the fed versus the fasted state, is less than about 100%, less than about 90%, less than about 80%, less than about 70%, less than about 60%, less than about 50%, less than about 40%, less than about 30%, less than about 20%, less than about 15%, less than about 10%, less than about 5%, less than about 3%, or essentially no difference.

Benefits of a dosage form which substantially eliminates the effect of food include an increase in subject convenience, thereby increasing subject compliance, as the subject does not need to ensure that they are taking a dose either with or without food.

D. Redispersibility Profiles of the Nanoparticulate Triamcinolone and Triamcinolone Derivative Compositions of the Invention

An additional feature of the nanoparticulate triamcinolone and triamcinolone derivative compositions of the invention is that the compositions redisperse such that the effective average particle size of the redispersed triamcinolone and triamcinolone derivative particles is less than about 2 microns. This is significant, because, if upon administration the nanoparticulate triamcinolone and triamcinolone derivative particles present in the compositions of the invention do not redisperse to a substantially nanoparticulate particle size, then the dosage form may lose the benefits afforded by formulating triamcinolone and triamcinolone derivatives into a nanoparticulate particle size.

This is because nanoparticulate triamcinolone and triamcinolone derivative compositions benefit from the small particle size; if the nanoparticulate triamcinolone and triamcinolone derivative particles do not redisperse into the small particle sizes upon administration, then “clumps” or agglomerated triamcinolone and triamcinolone derivative particles are formed. With the formation of such agglomerated particles, the bioavailability of the dosage form may fall.

Moreover, preferably the nanoparticulate triamcinolone and triamcinolone derivative compositions of the invention exhibit dramatic redispersion of the triamcinolone and triamcinolone derivative particles upon administration to a mammal, such as a human or animal, as demonstrated by reconstitution in a biorelevant aqueous media. Preferably, upon reconstitution in a biorelevant aqueous media, the compositions of the invention redisperse such that the effective average particle size of the redispersed triamcinolone and triamcinolone derivative particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed triamcinolone and triamcinolone derivative particles of the invention (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

E. Bioadhesive Nanoparticulate Triamcinolone and Triamcinolone Derivative Compositions

Bioadhesive nanoparticulate triamcinolone and triamcinolone derivative compositions of the invention comprise at least one cationic surface stabilizer, which are described in more detail below. Bioadhesive formulations of triamcinolone and triamcinolone derivatives exhibit exceptional bioadhesion to biological surfaces, such as mucous and mucous membranes.

In the case of bioadhesive nanoparticulate triamcinolone and triamcinolone derivative compositions, the term “bioadhesion” is used to describe the adhesion between the nanoparticulate triamcinolone and triamcinolone derivative compositions and a biological substrate (i.e., gastrointestinal mucin, lung tissue, nasal mucosa, etc.). See e.g., U.S. Pat. No. 6,428,814 for “Bioadhesive Nanoparticulate Compositions Having Cationic Surface Stabilizers,” which is specifically incorporated by reference.

The bioadhesive triamcinolone and triamcinolone derivative compositions of the invention are useful in any situation in which it is desirable to apply the compositions to a biological surface. The bioadhesive triamcinolone and triamcinolone derivative compositions preferably coat the targeted surface in a continuous and uniform film which is invisible to the naked human eye.

Bioadhesive nanoparticulate triamcinolone and triamcinolone derivative compositions slow the transit of the composition. As a result, some triamcinolone and triamcinolone derivative particles likely would adhere to the mucosa, prolonging exposure to the drug, thereby increasing absorption and the bioavailability of the administered dosage in situ.

F. Low Viscosity

Liquid dosage forms of conventional microcrystalline or non-nanoparticulate triamcinolone and triamcinolone derivatives can be expected to be a relatively large volume, viscous substance which may not be well accepted by patient populations. Moreover, viscous solutions can be problematic in parenteral administration because these solutions require a slow syringe push and can stick to tubing. Similarly, viscous solutions cannot be readily formulated into a fine and/or uniform mist for spray administration (e.g. nasal and topical spray). In addition, conventional formulations of poorly water-soluble active agents, such as triamcinolone and triamcinolone derivatives, tend to be unsafe for intravenous administration techniques, which are used primarily in conjunction with highly water-soluble substances.

Liquid dosage forms of the nanoparticulate triamcinolone and triamcinolone derivative compositions of the invention provide significant advantages over liquid dosage forms of conventional triamcinolone and triamcinolone derivatives microcrystalline compound. The low viscosity and silky texture of liquid dosage forms of the nanoparticulate triamcinolone and triamcinolone derivative compositions of the invention result in advantages in both preparation and use. These advantages include, for example: (1) better subject compliance due to the perception of a lighter formulation which is easier to consume and digest; (2) ease of dispensing because one can use a cup or a syringe; (3) potential for formulating a higher concentration of triamcinolone and triamcinolone derivatives resulting in a smaller dosage volume and thus less volume for the subject to consume or apply; and (4) easier overall formulation concerns.

Liquid triamcinolone and triamcinolone derivative dosage forms which are easier to consume are especially important when considering juvenile patients, terminally ill patients, and elderly patients. Viscous or gritty formulations, and those that require a relatively large dosage volume, are not well tolerated by these patient populations. Liquid oral dosage forms can be particularly preferably for patient populations who have difficulty consuming tablets, such as infants and the elderly.

The viscosities of liquid dosage forms of nanoparticulate triamcinolone and triamcinolone derivatives according to the invention are preferably less than about 1/200, less than about 1/175, less than about 1/150, less than about 1/125, less than about 1/100, less than about 1/75, less than about 1/50, or less than about 1/25 of a liquid oral dosage form of non-nanoparticulate triamcinolone and triamcinolone derivative compositions, at about the same concentration per ml of triamcinolone or triamcinolone derivative.

Typically the viscosity of liquid nanoparticulate triamcinolone or triamcinolone derivative dosage forms of the invention, at a shear rate of 0.1 (1/s), measured at about 20° C., is from about 2000 mPa s to about 1 mPa·s, from about 1900 mPa·s to about 1 mPa·s, from about 1800 mPa·s to about 1 mPa·s, from about 1700 mPa·s to about 1 mPa·s, from about 1600 mPa·s to about 1 mPa·s, from about 1500 mPa·s to about 1 mPa·s, from about 1400 mPa·s to about 1 mPa·s, from about 1300 mPa·s to about 1 mPa·s, from about 1200 mPa·s to about 1 mPa·s, from about 1100 mPa·s to about 1 mPa·s, from about 1000 mPa·s to about 1 mPa·s, from about 900 mPa·s to about 1 mPa·s, from about 800 mPa·s to about 1 mPa·s, from about 700 mPa·s to about 1 mPa·s, from about 600 mPa·s to about 1 mPa·s, from about 500 mPa·s to about 1 mPa·s, from about 400 mPa·s to about 1 mPa·s, from about 300 mPa·s to about 1 mPa·s, from about 200 mPa·s to about 1 mPa·s, from about 175 mPa·s to about 1 mPa·s, from about 150 mPa·s to about 1 mPa·s, from about 125 mPa·s to about 1 mPa·s, from about 100 mPa·s to about 1 mPa·s, from about 75 mPa·s to about 1 mPa·s, from about 50 mPa·s to about 1 mPa·s, from about 25 mPa·s to about 1 mPa·s, from about 15 mPa·s to about 1 mPa·s, from about 10 mPa·s to about 1 mPa·s, or from about 5 mPa·s to about 1 mPa·s. Such a viscosity is much more attractive for subject consumption and may lead to better overall subject compliance.

Viscosity is concentration and temperature dependent. Typically, a higher concentration results in a higher viscosity, while a higher temperature results in a lower viscosity. Viscosity as defined above refers to measurements taken at about 20° C. (The viscosity of water at 20° C. is 1 mPa·s.) The invention encompasses equivalent viscosities measured at different temperatures.

Another important aspect of the invention is that the nanoparticulate triamcinolone and triamcinolone derivative compositions of the invention, formulated into a liquid dosage form, are not turbid. “Turbid,” as used herein refers to the property of particulate matter that can be seen with the naked eye or that which can be felt as “gritty.” The nanoparticulate triamcinolone and triamcinolone derivative compositions of the invention, formulated into a liquid dosage form, can be poured out, extracted from a container, or sprayed through an atomizer as easily as water, whereas liquid dosage forms of non-nanoparticulate or solubilized triamcinolone and triamcinolone derivatives are expected to exhibit notably more “sluggish” characteristics.

The liquid formulations of this invention can be formulated for dosages in any volume but preferably equivalent or smaller volumes than a liquid dosage form of non-nanoparticulate triamcinolone and triamcinolone derivative compositions.

G. Sterile Filtered Nanoparticulate Triamcinolone and Triamcinolone Derivative Compositions

The nanoparticulate triamcinolone and triamcinolone derivative compositions of the invention can be sterile filtered. This obviates the need for heat sterilization, which can harm or degrade triamcinolone or a triamcinolone derivative, as well as result in crystal growth and particle aggregation.

Sterile filtration can be difficult because of the required small particle size of the composition. Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is normally not used to sterilize suspensions of micron-sized triamcinolone and triamcinolone derivatives because the triamcinolone and triamcinolone derivative particles are too large to pass through the membrane pores.

Sterile nanoparticulate triamcinolone and triamcinolone derivative dosage forms are particularly useful in treating immunocompromised patients, infants or juvenile patients, and the elderly, as these patient groups are the most susceptible to infection caused by a non-sterile liquid dosage form.

Because the nanoparticulate triamcinolone and triamcinolone derivative compositions of the invention, formulated into a liquid dosage form, can be sterile filtered, and because the compositions can have a very small effective average particle size, the compositions are suitable for parenteral administration.

H. Combination Pharmacokinetic Profile Compositions

In yet another embodiment of the invention, a first nanoparticulate triamcinolone or triamcinolone derivative composition providing a desired pharmacokinetic profile is co-administered, sequentially administered, or combined with at least one other triamcinolone or triamcinolone derivative composition that generates a desired different pharmacokinetic profile. More than two triamcinolone or triamcinolone derivative compositions can be co-administered, sequentially administered, or combined. While the first triamcinolone or triamcinolone derivative composition has a nanoparticulate particle size, the additional one or more triamcinolone or triamcinolone derivative compositions can be nanoparticulate, solubilized, or have a microparticulate particle size.

For example, a first triamcinolone or triamcinolone derivative composition can have a nanoparticulate particle size, conferring a short Tₘₐₓ and typically a higher Cₘₐₓ. This first triamcinolone or triamcinolone derivative composition can be combined, co-administered, or sequentially administered with a second composition comprising: (1) triamcinolone or a triamcinolone derivative having a larger (but still nanoparticulate as defined herein) particle size, and therefore exhibiting slower absorption, a longer Tₘₐₓ, and typically a lower Cₘₐₓ; or (2) a microparticulate or solubilized triamcinolone or triamcinolone derivative composition, exhibiting a longer Tₘₐₓ, and typically a lower Cₘₐₓ.

The second, third, fourth, etc., triamcinolone or triamcinolone derivative compositions can differ from the first, and from each other, for example: (1) in the effective average particle sizes of triamcinolone or triamcinolone derivative; or (2) in the dosage of triamcinolone or triamcinolone derivative. Such a combination composition can reduce the dose frequency required.

If the second triamcinolone or triamcinolone derivative composition has a nanoparticulate particle size, then preferably the triamcinolone or triamcinolone derivative particles of the second composition have at least one surface stabilizer associated with the surface of the drug particles. The one or more surface stabilizers can be the same as or different from the surface stabilizer(s) present in the first triamcinolone or triamcinolone derivative composition.

Preferably where co-administration of a “fast-acting” formulation and a “longer-lasting” formulation is desired, the two formulations are combined within a single composition, for example a dual-release composition.

I. Combination Active Agent Compositions

The invention encompasses the nanoparticulate triamcinolone and triamcinolone derivative compositions of the invention formulated or co-administered with one or more non-triamcinolone or triamcinolone derivative active agents. Methods of using such combination compositions are also encompassed by the invention. The non-triamcinolone or triamcinolone derivative active agents can be present in a crystalline phase, an amorphous phase, a semi-crystalline phase, a semi-amorphous phase, or a mixture thereof.

The compound to be administered in combination with nanoparticulate triamcinolone and triamcinolone derivative compositions of the invention can be formulated separately from the nanoparticulate triamcinolone or triamcinolone derivative composition or co-formulated with the nanoparticulate triamcinolone or triamcinolone derivative composition. Where a nanoparticulate triamcinolone or triamcinolone derivative composition is co-formulated with a second active agent, the second active agent can be formulated in any suitable manner, such as immediate-release, rapid-onset, sustained-release, or dual-release form.

Such non-triamcinolone or non-triamcinolone derivative active agents can be, for example, a therapeutic agent. A therapeutic agent can be a pharmaceutical agent, including a biologic. The active agent can be selected from a variety of known classes of drugs, including, for example, nutraceuticals, amino acids, proteins, peptides, nucleotides, anti-obesity drugs, central nervous system stimulants, carotenoids, corticosteroids, elastase inhibitors, anti-fungals, oncology therapies, anti-emetics, analgesics, cardiovascular agents, anti-inflammatory agents, such as NSAIDs and COX-2 inhibitors, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives (hypnotics and neuroleptics), astringents, alpha-adrenergic receptor blocking agents, beta-adrenoceptor blocking agents, blood products and substitutes, cardiac inotropic agents, contrast media, corticosteroids, cough suppressants (expectorants and mucolytics), decongestants, diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

Examples of representative active agents useful in this invention include, but are not limited to, acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

A description of these classes of active agents and a listing of species within each class can be found in Martindale's The Extra Pharmacopoeia, 31^{st} Edition (The Pharmaceutical Press, London, 1996), specifically incorporated by reference. The active agents are commercially available and/or can be prepared by techniques known in the art.

Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., arginine, iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.”

Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. Dietary supplements and nutraceuticals are also disclosed in Physicians' Desk Reference for Nutritional Supplements, 1st Ed. (2001) and The Physicians' Desk Reference for Herbal Medicines, 1st Ed. (2001), both of which are also incorporated by reference. A nutraceutical or dietary supplement, also known as a phytochemical or functional food, is generally any one of a class of dietary supplements, vitamins, minerals, herbs, or healing foods that have medical or pharmaceutical effects on the body.

In a particularly preferred embodiment, nanoparticulate triamcinolone and triamcinolone derivative compositions are combined with at least one antihistamine. Useful antihistamines include, for example, fexofenadine, azelastine, hydoxyzine, diphenhydramine, loratadine, chlorpheniramine maleate, cyproheptadine, promethazine, phenylephrine tannate, acrivastine, and cetirizine.

In a further particularly preferred embodiment, nanoparticulate triamcinolone and triamcinolone derivative compositions are combined with at least one decongestant. Useful decongestants include, for example, pseudoephedrine, oxymetazoline, xylometazoline, naphazoline, naphazoline, and tetrahydrozoline.

In an additional preferred embodiment, nanoparticulate triamcinolone and triamcinolone derivative compositions are combined with at least one bronchodilator, such as short-acting and long-acting agonists, anticholinergics, and theophylline. Useful short-acting beta2-agonists include pirbuterol and albuterol. Long-acting beta2-agonists include formoteroll, salmeterol and albuterol. Useful anticholinergics include ipratropium bromide.

In yet another embodiment, the compositions of the invention are combined with an anti-fungal agent, such as amphotericin B, nystatin, fluconazole, ketoconazole, terbinafine, itraconazole, imidazole, triazole, ciclopirox, clotrimazole, and miconazole.

Finally, in a preferred embodiment of the invention, the compositions of the invention can be combined with an immunosuppressant, such as for treatment required following organ transplantation.

J. Miscellaneous Benefits of the Nanoparticulate Triamcinolone and Triamcinolone Derivative Compositions of the Invention

The nanoparticulate triamcinolone and triamcinolone derivative compositions preferably exhibit an increased rate of dissolution as compared to microcrystalline or non-nanoparticulate forms of triamcinolone or triamcinolone derivatives. In addition, the nanoparticulate triamcinolone and triamcinolone derivative compositions preferably exhibit improved performance characteristics for oral, intravenous, subcutaneous, or intramuscular injection, such as higher dose loading and smaller tablet or liquid dose volumes. Moreover, the nanoparticulate triamcinolone and triamcinolone derivative compositions of the invention do not require organic solvents or pH extremes.

II. Triamcinolone and Triamcinolone Derivative Compositions

The invention provides compositions comprising nanoparticulate triamcinolone and triamcinolone derivative particles and at least one surface stabilizer. The surface stabilizers are preferably associated with the surface of the triamcinolone or triamcinolone derivative particles. Surface stabilizers useful herein do not chemically react with the triamcinolone and triamcinolone derivative particles or itself. Preferably, individual molecules of the surface stabilizer are essentially free of intermolecular cross-linkages. The compositions can comprise two or more surface stabilizers.

The present invention also includes nanoparticulate triamcinolone and triamcinolone derivative compositions together with one or more non-toxic physiologically acceptable carriers, adjuvants, or vehicles, collectively referred to as carriers. The compositions can be formulated for parenteral injection (i.e., intravenous, intramuscular, or subcutaneous), oral administration (in solid, liquid, or aerosol (i.e., pulmonary or nasal form), vaginal, nasal, rectal, ocular, local (powders, creams, ointments or drops), buccal, intracisternal, intraperitoneal, topical administration, and the like.

A. Triamcinolone and Triamcinolone Derivatives

As used herein, “triamcinolone” refers to ((11β, 16 [symbol] )-9-fluoro-11, 17, 18, 21-dihydroxy-pregna-1,4-diene-3,20-dione or a salt thereof having the following chemical structure:

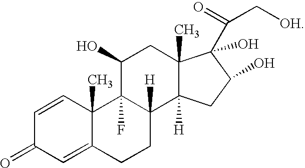

“Triamcinolone derivatives” refers to any chemical derivative of triamcinolone and includes, but is not limited to, triamcinolone acetonide ((11β,16α)-9-fluoro-11,21-dihydroxy-16,17-[1-methylethylidenebis(oxy)]-pregna-1,4-diene-3,20-dione) having following chemical structure:

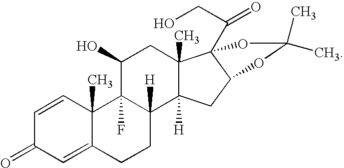

“Triamcinolone derivatives” also includes, but is not limited to, triamcinolone diacetate ((11β,16α)-16,21-bis(acetyloxy)-9-fluoro-11,17-dihydroxypregna-1,4-diene-3,20-dione) having the following chemical structure:

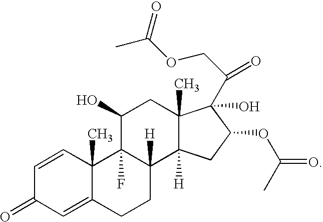

“Triamcinolone derivatives” includes, but is not limited to, triamcinolone hexacetonide ((11β,16α)-21-(3,3dimethyl-1-oxobutoxy)-9-fluoro-11-hydroxy-16,17-[(1-methylethylidene)bis(oxy)]-pregna-1,4-diene-3,20-dione), having the following chemical structure:

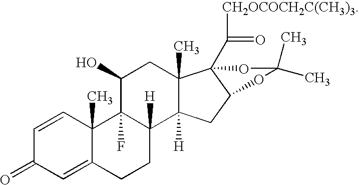

Finally, “triamcinolone derivatives” includes, but is not limited to, triamcinolone benetonide ((11β,16α)-21-[3-(benzoylamino)-2-methyl-1-oxopropoxy]-9-fluoro-11-hydroxy-16,17-[(1-methylethylidene)bis(oxy)]-pregna-1,4-diene-3,20-dione) having the following chemical structure:

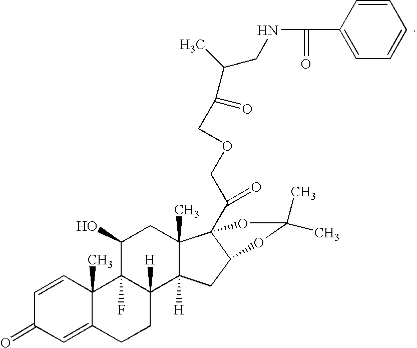

Triamcinolone and triamcinolone derivatives can be in a crystalline phase, an amorphous phase, a semi-crystalline phase, a semi-amorphous phase, or a mixtures thereof.

B. Surface Stabilizers

The choice of a surface stabilizer for triamcinolone and triamcinolone derivatives is non-trivial and requires extensive experimentation to realize a desirable formulation. Accordingly, the present invention is directed to the surprising discovery that triamcinolone and triamcinolone derivative nanoparticulate compositions can be made.

Combinations of more than one surface stabilizer can be used in the invention. Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, cationic, zwitterionic, and ionic surfactants.

Representative examples of other useful surface stabilizers include hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-IOG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

Depending upon the desired method of administration, bioadhesive formulations of nanoparticulate triamcinolone and triamcinolone derivatives can be prepared by selecting one or more cationic surface stabilizers that impart bioadhesive properties to the resultant composition. Useful cationic surface stabilizers are described below.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt) (also known as DPPE-PEG(2000)-Amine Na) (Avanti Polar Lipids, Alabaster, Ala.), Poly(2-methacryloxyethyl trimethylammonium bromide) (Polysciences, Inc., Warrington, Pa.) (also known as S1001), poloxamines such as Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.), lysozyme, long-chain polymers such as alginic acid, carrageenan (FMC Corp.), and POLYOX (Dow, Midland, Mich.).

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl(ethenoxy)₄ ammonium chloride or bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₆, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyidimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric cationic surface stabilizers are any nonpolymeric compound, such as benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R4 is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₆(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

The surface stabilizers are commercially available and/or can be prepared by techniques known in the art.

Preferred surface stabilizers include, but are not limited to, a random copolymer of vinyl pyrrolidone and vinyl acetate, such as Plasdone® S-630 (ISP Technologies, Inc.), sodium lauryl sulfate (SLS), lysozyme, tyloxapol, and combinations thereof.

C. Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

D. Nanoparticulate Triamcinolone and Triamcinolone Derivative Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The compositions of the invention comprise triamcinolone and/or triamcinolone derivative particles which have an effective average particle size of less than about 2000 nm (i.e., 2 microns), less than about 1900 nm, less than less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, or less than about 50 nm, when measured by the above-noted techniques.

By “an effective average particle size of less than about 2000 nm” it is meant that at least 50% of the nanoparticulate triamcinolone and/or triamcinolone derivative particles have a weight average particle size of less than about 2000 nm, when measured by the above-noted techniques. In other embodiments of the invention, at least about 70%, at least about 90%, at least about 95%, or at least about 99% of the nanoparticulate triamcinolone and triamcinolone and/or triamcinolone derivative particles have a particle size of less than the effective average, by weight, i.e., less than about 2000 nm, less than about 1900 nm, less than less than about 1800 nm, less than about 1700 nm, etc.

If the nanoparticulate triamcinolone or triamcinolone derivative composition is combined with a microparticulate triamcinolone or triamcinolone derivative, or microparticulate non-triamcinolone or non-triamcinolone derivative active agent composition, then such a composition is either solubilized or has an effective average particle size of greater than about 2 microns. By “an effective average particle size of greater than about 2 microns” it is meant that at least 50% of the microparticulate triamcinolone or triamcinolone derivative, or non-triamcinolone or non-triamcinolone derivative, particles have a particle size of greater than about 2 microns, by weight, when measured by the above-noted techniques. In other embodiments of the invention, at least about 70%, at least about 90%, at least about 95%, or at least about 99%, by weight, of the microparticulate triamcinolone or triamcinolone derivative, or non-triamcinolone or non-triamcinolone derivative, particles have a particle size greater than about 2 microns.

In the present invention, the value for D50 of a nanoparticulate triamcinolone or triamcinolone derivative composition is the particle size below which 50% of the triamcinolone or triamcinolone derivative particles fall, by weight. Similarly, D90, D95, and D99 are the particle sizes below which 90%, 95%, and 99%, respectively, of the triamcinolone or triamcinolone derivative particles fall, by weight.

E. Concentration of Nanoparticulate Triamcinolone and Triamcinolone Derivatives and Surface Stabilizers

The relative amounts of triamcinolone or a triamcinolone derivative and one or more surface stabilizers can vary widely. The optimal amount of the individual components can depend, for example, upon the particulate triamcinolone selected, and the hydrophilic lipophilic balance (HLB), melting point, and the surface tension of water solutions of the stabilizer, etc.

The concentration of triamcinolone or a triamcinolone derivative can vary from about 99.5% to about 0.001%, from about 95% to about 0.1%, or from about 90% to about 0.5%, by weight, based on the total combined dry weight of the triamcinolone or triamcinolone derivative and at least one surface stabilizer, not including other excipients.

The concentration of at least one surface stabilizer can vary from about 0.5% to about 99.999%, from about 5.0% to about 99.9%, or from about 10% to about 99.5%, by weight, based on the total combined dry weight of the triamcinolone or triamcinolone derivative and at least one surface stabilizer, not including other excipients.

III. Methods of Making Nanoparticulate Triamcinolone and Triamcinolone Derivatives Formulations

The nanoparticulate triamcinolone and triamcinolone derivative compositions can be made using, for example, milling, homogenization, or precipitation techniques. Exemplary methods of making nanoparticulate active agent compositions are described in the '684 patent. Methods of making nanoparticulate active agent compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

Following milling, homogenization, precipitation, etc., the resultant nanoparticulate triamcinolone or triamcinolone derivative composition can be utilized in solid or liquid dosage formulations, such as controlled release formulations, solid dose fast melt formulations, aerosol formulations, nasal formulations, lyophilized formulations, tablets, capsules, solid lozenge, powders, creams, ointments, etc.

A. Milling to Obtain Nanoparticulate Triamcinolone and Triamcinolone Derivatives Dispersions

Milling triamcinolone and/or a triamcinolone derivative to obtain a nanoparticulate dispersion comprises dispersing triamcinolone and/or a triamcinolone derivative in a liquid dispersion media in which the triamcinolone and/or triamcinolone derivative is poorly soluble, followed by applying mechanical means in the presence of grinding media to reduce the particle size of the triamcinolone and/or triamcinolone derivative to the desired effective average particle size. The dispersion media can be, for example, water, safflower oil, ethanol, t-butanol, glycerin, polyethylene glycol (PEG), hexane, or glycol. Water is a preferred dispersion media.

The triamcinolone and/or triamcinolone derivative particles can be reduced in size in the presence of at least one surface stabilizer. Alternatively, the triamcinolone and/or triamcinolone derivative particles can be contacted with one or more surface stabilizers after attrition. Other compounds, such as a diluent, can be added to the triamcinolone or triamcinolone derivative/surface stabilizer composition during the size reduction process. Dispersions can be manufactured continuously or in a batch mode.

B. Precipitation to Obtain Nanoparticulate Triamcinolone and Triamcinolone Derivatives Compositions

Another method of forming the desired nanoparticulate triamcinolone and/or triamcinolone derivative composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving triamcinolone and/or a triamcinolone derivative in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means. Dispersions can be manufactured continuously or in a batch mode.

C. Homogenization to Obtain Nanoparticulate Triamcinolone and/or Triamcinolone Derivative Compositions

Exemplary homogenization methods of preparing nanoparticulate active agent compositions are described in U.S. Pat. No. 5,510,118, for “Process of Preparing Therapeutic Compositions Containing Nanoparticles.”

Such a method comprises dispersing particles of triamcinolone and/or a triamcinolone derivative in a liquid dispersion media in which the particles are poorly soluble, followed by subjecting the dispersion to homogenization to reduce the particle size of the triamcinolone and/or triamcinolone derivative to the desired effective average particle size. The dispersion media can be, for example, water, safflower oil, ethanol, t-butanol, glycerin, polyethylene glycol (PEG), hexane, or glycol. Water is a preferred dispersion media.

The triamcinolone and/or triamcinolone derivative particles can be reduced in size in the presence of at least one surface stabilizer. Alternatively, the triamcinolone and triamcinolone derivative particles can be contacted with one or more surface stabilizers either before or after attrition. Other compounds, such as a diluent, can be added to the triamcinolone or triamcinolone derivative/surface stabilizer composition either before, during, or after the size reduction process. Dispersions can be manufactured continuously or in a batch mode.

IV. Methods of Using Nanoparticulate Triamcinolone and Triamcinolone Derivative Formulations

The method of the invention comprises administering to a subject an effective amount of a composition comprising nanoparticulate triamcinolone and/or a triamcinolone derivative. The triamcinolone and/or triamcinolone derivative compositions of the present invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), or as a buccal or nasal spray.

As used herein, the term “subject” is used to mean an animal, preferably a mammal, including a human or non-human. The terms patient and subject may be used interchangeably.

The compositions of the invention are useful in treating conditions or disorders where a glucocorticoid is typically used. In addition, the compositions of the invention are useful in treating conditions or disorders where a steroidal anti-inflammatory agent is typically used, such as in treat swelling, heat, redness, and/or pain. The compositions of the invention can also be administered with other drugs to prevent rejection of transplanted organs and to treat certain types of cancer.

Exemplary conditions or disorders that can be treated with the compositions of the invention include, but are not limited to, arthritis, skin disorders, blood disorders, kidney disorders, eye disorders, thyroid disorders, intestinal disorders, allergies, asthma, bronchial asthma, cancer, neoplastic diseases, tendinitis, allergic reactions, seasonal allergic rhinitis, perennial allergic rhinitis, oral inflammation, oral lesions, oral ulcers, bursitis, epicondylitis, keloids, endocrine disorders, herpes zoster ophthalmicus, hemolytic anemia, and acute rheumatic carditis.

Exemplary skin disorders that can be treated with the compositions of the invention include, but are not limited to, contact dermatitis, atopic dermatitis, psoriasis, eczema, and general dermatitis. Exemplary arthritic conditions that can be treated with the compositions of the invention include, but are not limited to, osteoarthritis, acute nonspecific osteoarthritis, posttraumatic osteoarthritis, and rheumatoid arthritis. Exemplary intestinal disorders that can be treated with the compositions of the invention include, but are not limited to, ulcerative colitis, colitis, gastroenteritis, irritable bowel disorder, and Crohn's disease. Exemplary types of cancer or neoplastic diseases that can be treated with the compositions of the invention include, but are not limited to, lupus, leukemias, and lymphoma.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, powder aerosols, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable aerosols, emulsions, solutions, suspensions, syrups, and elixirs. In addition to the active agent, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

One of ordinary skill will appreciate that effective amounts of triamcinolone and triamcinolone derivatives can be determined empirically and can be employed in pure form or, where such forms exist, in pharmaceutically acceptable salt, ester, or prodrug form. Actual dosage levels of triamcinolone and triamcinolone derivative in the nanoparticulate compositions of the invention may be varied to obtain an amount of triamcinolone or triamcinolone derivative that is effective to obtain a desired therapeutic response for a particular composition and method of administration. The selected dosage level therefore depends upon the desired therapeutic effect, the route of administration, the potency of the administered triamcinolone or triamcinolone derivative, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### EXAMPLE 1

The purpose of this examples was to prepare dispersions of nanoparticulate triamcinolone acetonide, and to test the prepared compositions for stability. Stability was determined by static light scattering methods to verify whether or not larger crystals of triamcinolone acetonide formed.

A nanoparticulate colloidal dispersion (NCD) of triamcinolone acetonide having 5% (w/w) triamcinolone acetonide and 0.5% (w/w) tyloxapol was milled for 1 hour under high energy milling conditions in a DYN®-Mill KDL (Willy A. Bachofen AG, Maschinenfabrik, Basel, Switzerland) equipped with a 150 cc batch milling chamber and using 500 μm polymeric attrition media (Dow Chemical, Midland Mich.).

The final (weight) mean particle size of the triamcinolone acetonide particles was 182 nm, with D50<173 nm, D90<262 nm, and D95<296 nm, as measured using a Horiba LA-910 Laser Scattering Particle Size Distribution Analyzer (Horiba Instruments, Irvine, Calif.) and a 0.01% w/w solution of benzalkonium chloride as the dispersing medium.

The average particle size of the triamcinolone acetonide dispersion increased by 54 nm to 236 nm, with D50<225 nm, D90<325 nm, and D95<364 nm, after storage at room temperature for 24 hours, as shown in Table 1. Particle size was measured using a Horiba LA-910 Laser Scattering Particle Size Distribution Analyzer (Horiba Instruments, Irvine, Calif.).

TABLE 1

| | Triamcinolone | | Initial | Dₘₑₐₙ after |
|---|---|---|---|---|
| Sample Ref. | Acetonide | tyloxapol | Dₘₑₐₙ | 24 hrs storage |
| Sample 1 | 5.0% | 0.5% | 182 nm | 236 nm |

This example demonstrates that nanoparticulate triamcinolone compositions can be made, and that compositions having tyloxapol as a surface stabilizer may exhibit slight particle size growth over time.

### EXAMPLE 2

The purpose of this example was to prepare dispersions of nanoparticulate triamcinolone acetonide, and to test the prepared compositions for stability. Stability was determined by static light scattering methods to verify whether or not larger crystals of triamcinolone or triamcinolone derivative formed.

A nanoparticulate colloidal dispersion (NCD) of triamcinolone acetonide having 5% (w/w) triamcinolone acetonide, 0.5% (w/w) tyloxapol, and 0.5% (w/w) sodium chloride as a crystal growth inhibitor was milled for 2 hours under high energy milling conditions in a DYNO®-Mill KDL (Willy A. Bachofen AG, Maschinenfabrik, Basel, Switzerland) equipped with a 150 cc batch milling chamber and using 500 μm polymeric attrition media (Dow Chemical, Midland, Mich.).

The final (weight) mean particle size of the triamcinolone acetonide particles was 149 nm, with D90<212 nm, as measured using a Horiba LA-910 Laser Scattering Particle Size Distribution Analyzer (Horiba Instruments, Irvine, Calif.) and a 0.5% w/w solution of sodium chloride as the dispersing medium.

In the presence of 0.5% w/w sodium chloride as a crystal growth inhibitor, the average particle size of the triamcinolone acetonide dispersion increased by only 16 nm to 165 nm (D90<243 nm) after storage at room temperature for 24 h as shown in Table 2.

TABLE 2

| Sample | Triamcinolone | | | Initial | Dₘₑₐₙ after |
|---|---|---|---|---|---|
| Ref. | Acetonide | tyloxapol | NaCl | Dₘₑₐₙ | 24 hrs storage |
| Sample 2 | 5% | 0.5% | 0.5% | 149 nm | 165 nm |

The results of Examples 1 and 2 show that nanoparticulate triamcinolone compositions can be made. In addition, the results of Examples 1 and 2 demonstrate that nanoparticulate triamcinolone acetonide compositions having tyloxapol as a surface stabilizer may benefit from the addition of a crystal growth inhibitor, such as sodium chloride.

### EXAMPLE 3

The purpose of this example was to prepare a nanoparticulate triamcinolone acetonide composition using lysozyme as a surface stabilizer.

Lysozyme, also known as muramidase, N-acetylmuramylhydrolase, and globulin G1, has a molecular weight of about 14,400. It is a mucolytic enzyme with antibiotic properties first discovered by A. Fleming, Proc. Roy. Soc. London, 93B:306 (1922). Although lysozyme has antibiotic properties, it is a large molecule that is not particularly useful as a drug. It can be applied topically, but cannot rid the entire body of disease because it is too large to travel between cells.

An aqueous dispersion of 1% (w/w) lysozyme and 5% (w/w) triamcinolone acetonide was charged into a NanoMill™ (Elan Drug Delivery) equipped with a 10 cc batch chamber. The mill speed was 2500 rpm, and the temperature during milling was maintained at 5° C. The triamcinolone acetonide/lysozyme mixture was milled for 30 min.

Following milling, the mean particle size, D50, and D90 were measured for the milled triamcinolone acetonide composition using a Horiba LA-910 Laser Scattering Particle Size Distribution Analyzer (Horiba Instruments, Irvine, Calif.). The milled triamcinolone acetonide composition was also evaluated via a microscope to detect any aggregation. The results are shown below in Table 3.

TABLE 3

| | Mean | D50 | D90 | | Mill Time |
|---|---|---|---|---|---|
| Compound | (nm) | (nm) | (nm) | Microscope | (hr) |
| Triamcinolone | 114 | 107 | 172 | Stable | 0.5 |
| acetomide | | | | | |

The results demonstrate that stable nanoparticulate triamcinolone compositions can be made.

### EXAMPLE 4

The purpose of this example was to prepare a nanoparticulate triamcinolone acetonide composition comprising a copolymer of vinyl pyrrolidone and vinyl acetate and sodium lauryl sulfate as surface stabilizers.

An aqueous solution of 1% (w/w) Plasdone® S-630 (60% vinyl pyrrolidone and 40% vinyl acetate) (ISP Technologies, Inc.) and 0.05% (w/w) sodium lauryl sulfate (SLS) (Spectrum) was prepared by dissolving 0.85 g of polymer and 4.30 g of a 1% SLS solution in 76.10 g of deionized water. The stabilizer solution was mixed with 4.26 g of triamcinolone acetonide (5% w/w) and charged into the chamber of a DYNO®-Mill Type KDL media mill (Willy Bachofen AG, Basel, Switzerland) along with 500 micron polymeric media (PolyMill® 500; Dow Chemical, Midland, Mich.). The mill was operated for 2 hours.

Upon completion of milling, the milled triamcinolone acetonide particles had a mean particle size of 121 nm, with a D90 of 194 nm, as measured using a Horiba LA-910 Laser Scattering Particle Size Distribution Analyzer (Horiba Instruments, Irvine, Calif.).

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A composition comprising:
(a) particles of at least one triamcinolone or a salt thereof, wherein the triamcinolone particles have an effective average particle size of less than about 2000 nm; and
(b) at least one surface stabilizer.

2. The composition of claim 1, wherein the triamcinolone is selected from the group consisting of triamcinolone, triamcinolone acetonide, triamcinolone diacetate, triamcinolone hexacetonide, and triamcinolone benetonide.

3. The composition of claim 2, wherein the triamcinolone is triamcinolone acetonide.

4. The composition of claim 1, wherein the triamcinolone is selected from the group consisting of a crystalline phase, an amorphous phase, a semi-crystalline phase, a semi-amorphous phase, and mixtures thereof.

5. The composition of claim 1, wherein the effective average particle size of the triamcinolone particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

6. The composition of claim 1, wherein the composition is formulated for administration selected from the group consisting of oral, pulmonary, rectal, ophthalmic, colonic, parenteral, intracisternal, intravaginal, intraperitoneal, local, buccal, nasal, and topical administration.

7. The composition of claim 1 formulated into a dosage form selected from the group consisting of liquid dispersions, sachets, lozenges, oral suspensions, gels, aerosols, ointments, creams, tablets, capsules, and powders.

8. The composition of claim 1 formulated into a dosage form selected from the group consisting of controlled release formulations, fast melt formulations, lyophilized formulations, delayed release formulations, extended release formulations, pulsatile release formulations, and mixed immediate release and controlled release formulations.

9. The composition of claim 1, wherein the composition further comprises one or more pharmaceutically acceptable excipients, carriers, or a combination thereof.

10. The composition of claim 1, wherein the triamcinolone or a salt thereof is present in an amount selected from the group consisting of from about 99.5% to about 0.001%, from about 95% to about 0.1%, and from about 90% to about 0.5%, by weight, based on the total combined dry weight of the triamcinolone or salt thereof and at least one surface stabilizer, not including other excipients.

11. The composition of claim 1, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the triamcinolone or salt thereof and at least one surface stabilizer, not including other excipients.

12. The composition of claim 1, comprising at least two surface stabilizers.

13. The composition of claim 1, wherein the surface stabilizer is selected from the group consisting of an anionic surface stabilizer, a cationic surface stabilizer, a zwitterionic surface stabilizer, and an ionic surface stabilizer.

14. The composition of claim 13, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, and random copolymers of vinyl acetate and vinyl pyrrolidone.

15. The composition of claim 13, wherein the at least one cationic surface stabilizer is selected from the group consisting of a polymer, a biopolymer, a polysaccharide, a cellulosic, an alginate, a nonpolymeric compound, and a phospholipid.

16. The composition of claim 13, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl (ethenoxy)₄ ammonium bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyidimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

17. The composition of claim 1, comprising as a surface stabilizer a random copolymer of vinyl pyrrolidone and vinyl acetate, sodium lauryl sulfate, lysozyme, tyloxapol, or a combination thereof.

18. The composition of any of claims 13, 15, 16, or 17, wherein the composition is bioadhesive.

19. The composition of claim 1, further comprising at least one additional triamcinolone composition having an effective average particle size which is different that the effective average particle size of the triamcinolone composition of claim 1.

20. The composition of claim 1, additionally comprising one or more non-triamcinolone active agents.

21. The composition of claim 20, wherein said additional one or more non-triamcinolone active agents are selected from the group consisting of nutraceuticals, amino acids, proteins, peptides, nucleotides, anti-obesity drugs, central nervous system stimulants, carotenoids, corticosteroids, elastase inhibitors, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, alpha-adrenergic receptor blocking agents, beta-adrenoceptor blocking agents, blood products, blood substitutes, cardiac inotropic agents, contrast media, corticosteroids, cough suppressants, decongestants, diagnostic agents, diagnostic imaging agents, diuretics, dopaminergics, haemostatics, immunological agents, lipid regulating agents, muscle relaxants, parasympathomimetics, parathyroid calcitonin, parathyroid biphosphonates, prostaglandins, radio-pharmaceuticals, sex hormones, anti-allergic agents, stimulants, anoretics, sympathomimetics, thyroid agents, vasodilators, and xanthines.

22. The composition of claim 20, wherein said additional one or more non-triamcinolone active agents are selected from the group consisting of acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

23. The composition of claim 20, further comprising at least one antihistamine, decongestant, bronchodilator, anti-fungal, anti-cancer agent, or immunosuppressant.

24. The composition of claim 23, wherein the antihistamine is selected from the group consisting of fexofenadine, azelastine, hydoxyzine, diphenhydramine, loratadine, chlorpheniramine maleate, cyproheptadine, promethazine, phenylephrine tannate, acrivastine, and cetirizine.

25. The composition of claim 23, wherein the decongestant is selected from the group consisting of pseudoephedrine, oxymetazoline, xylometazoline, naphazoline, naphazoline, and tetrahydrozoline.

26. The composition of claim 23, wherein the brochodilator is selected from the group consisting of short-acting beta2-agonists, long-acting beta2-agonists, anticholinergics, and theophyllines.

27. The composition of claim 23, wherein the anti-fungal agent is selected from the group consisting of amphotericin B, nystatin, fluconazole, ketoconazole, terbinafine, itraconazole, imidazole, triazole, ciclopirox, clotrimazole, and miconazole.

28. The composition of claim 1, wherein upon administration to a mammal the triamcinolone particles redisperse such that the particles have an effective average particle size of less than about 2 microns.

29. The composition of claim 28, wherein upon administration the composition redisperses such that the triamcinolone particles have an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

30. The composition of claim 1, wherein the composition redisperses in a biorelevant media such that the triamcinolone particles have an effective average particle size of less than about 2 microns.

31. The composition of claim 30, wherein the biorelevant media is selected from the group consisting of water, aqueous electrolyte solutions, aqueous solutions of a salt, aqueous solutions of an acid, aqueous solutions of a base, and combinations thereof.

32. The composition of claim 30, wherein the composition redisperses in a biorelevant media such that the triamcinolone particles have an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

33. The composition of claim 1, wherein the Tₘₐₓ of the triamcinolone composition, when assayed in the plasma of a mammalian subject following administration, is less than the Tₘₐₓ exhibited by a non-nanoparticulate composition of the same triamcinolone, administered at the same dosage.

34. The composition of claim 33, wherein the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate composition of the same triamcinolone, administered at the same dosage.

35. The composition of claim 1, wherein the Cₘₐₓ of the triamcinolone composition, when assayed in the plasma of a mammalian subject following administration, is greater than the Cₘₐₓ exhibited by a non-nanoparticulate composition of the same triamcinolone, administered at the same dosage.

36. The composition of claim 35, wherein the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate composition of the same triamcinolone, administered at the same dosage.

37. The composition of claim 1, wherein the AUC of the triamcinolone composition, when assayed in the plasma of a mammalian subject following administration, is greater than the AUC exhibited by a non-nanoparticulate composition of the same triamcinolone, administered at the same dosage.

38. The composition of claim 37, wherein the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by a non-nanoparticulate composition of the same triamcinolone, administered at the same dosage.

39. The composition of claim 1 which does not produce significantly different absorption levels when administered under fed as compared to fasting conditions.

40. The composition of claim 39, wherein the difference in absorption of the triamcinolone composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

41. The composition of claim 1, wherein administration of the composition to a human in a fasted state is bioequivalent to administration of the composition to a subject in a fed state.

42. The composition of claim 41, wherein “bioequivalency” is established by a 90% confidence Interval of between 0.80 and 1.25 for both Cₘₐₓ and AUC.

43. The composition of claim 41, wherein “bioequivalency” is established by a 90% confidence Interval of between 0.80 and 1.25 for AUC and a 90% confidence Interval of between 0.70 to 1.43 for Cₘₐₓ.

44. The composition of claim 1 formulated into a liquid dosage form, wherein the dosage form has a viscosity of less than about 2000 mPa·s, measured at 20° C., at a shear rate of 0.1 (1/s).

45. The composition of claim 44, having a viscosity at a shear rate of 0.1 (1/s), measured at 20° C., selected from the group consisting of from about 2000 mPa·s to about 1 mPa·s, from about 1900 mPa·s to about 1 mPa·s, from about 1800 mPa·s to about 1 mPa·s, from about 1700 mPa·s to about 1 mPa·s, from about 1600 mPa·s to about 1 mPa·s, from about 1500 mPa·s to about 1 mPa·s, from about 1400 mPa·s to about 1 mPa·s, from about 1300 mPa·s to about 1 mPa·s, from about 1200 mPa·s to about 1 mPa·s, from about 1100 mPa·s to about 1 mPa·s, from about 1000 mPa·s to about 1 mPa·s, from about 900 mPa·s to about 1 mPa·s, from about 800 mPa·s to about 1 mPa·s, from about 700 mPa·s to about 1 mPa·s, from about 600 mPa·s to about 1 mPa·s, from about 500 mPa·s to about 1 mPa·s, from about 400 mPa·s to about 1 mPa·s, from about 300 mPa·s to about 1 mPa·s, from about 200 mPa·s to about 1 mPa·s, from about 175 mPa·s to about 1 mPa·s, from about 150 mPa·s to about 1 mPa·s, from about 125 mPa·s to about 1 mPa·s, from about 100 mPa·s to about 1 mPa·s, from about 75 mPa·s to about 1 mPa·s, from about 50 mPa·s to about 1 mPa·s, from about 25 mPa·s to about 1 mPa·s, from about 15 mPa·s to about 1 mPa·s, from about 10 mPa·s to about 1 mPa·s, and from about 5 mPa·s to about 1 mPa·s.

46. The composition of claim 44, wherein the viscosity of the dosage form is selected from the group consisting of less than about 1/200, less than about 1/100, less than about 1/50, less than about 1/25, and less than about 1/10 of the viscosity of a liquid dosage form of a non-nanoparticulate composition of the same triamcinolone, at about the same concentration per ml of triamcinolone.

47. The composition of claim 44, wherein the viscosity of the dosage form is selected from the group consisting of less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, and less than about 90% of the viscosity of a liquid dosage form of a non-nanoparticulate composition of the same triamcinolone, at about the same concentration per ml of triamcinolone.

48. A method of making a triamcinolone composition comprising contacting particles of a triamcinolone or a salt thereof with at least one surface stabilizer for a time and under conditions sufficient to provide a triamcinolone composition having an effective average particle size of less than about 2000 nm.

49. The method of claim 48, wherein the triamcinolone is selected from the group consisting of triamcinolone, triamcinolone acetonide, triamcinolone diacetate, triamcinolone hexacetonide, and triamcinolone benetonide.

50. The method of claim 49, wherein the triamcinolone is triamcinolone acetonide.

51. The method of claim 48, wherein said contacting comprises grinding.

52. The method of claim 51, wherein said grinding comprises wet grinding.

53. The method of claim 48, wherein said contacting comprises homogenizing.

54. The method of claim 48, wherein said contacting comprises:
(a) dissolving the particles of a triamcinolone or salt thereof in a solvent;
(b) adding the resulting triamcinolone solution to a solution comprising at least one surface stabilizer; and
(c) precipitating the solubilized triamcinolone/surface stabilizer composition by the addition of a non-solvent.

55. The method of claim 48, wherein the triamcinolone or salt thereof is selected from the group consisting of a crystalline phase, an amorphous phase, a semi-crystalline phase, a semi-amorphous phase, and mixtures thereof.

56. The method of claim 48, wherein the effective average particle size of the triamcinolone particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

57. The method of claim 48, wherein the triamcinolone or salt thereof is present in an amount selected from the group consisting of from about 99.5% to about 0.001%, from about 95% to about 0.1%, and from about 90% to about 0.5%, by weight, based on the total combined dry weight of the triamcinolone or a salt thereof and at least one surface stabilizer, not including other excipients.

58. The method of claim 48, wherein the at least one surface stabilizer is present in an amount selected from the group consisting of from about 0.5% to about 99.999%, from about 5.0% to about 99.9%, and from about 10% to about 99.5% by weight, based on the total combined dry weight of the triamcinolone or a salt thereof and at least one surface stabilizer, not including other excipients.

59. The method of claim 48, utilizing at least two surface stabilizers.

60. The method of claim 48, wherein the surface stabilizer is selected from the group consisting of an anionic surface stabilizer, a cationic surface stabilizer, a zwitterionic surface stabilizer, and an ionic surface stabilizer.

61. The method of claim 60, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

62. The method of claim 60, wherein the at least one cationic surface stabilizer is selected from the group consisting of a polymer, a biopolymer, a polysaccharide, a cellulosic, an alginate, a nonpolymeric compound, and a phospholipid.

63. The method of claim 60, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl(ethenoxy)₄ ammonium bromide, N-alkyl (C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts;
amines, amine salts, amine oxides, imide azolinium salts, protonated quaternary acrylamides, methylated quaternary polymers, and cationic guar.

64. The composition of claim 1, comprising as a surface stabilizer a random copolymer of vinyl pyrrolidone and vinyl acetate, sodium lauryl sulfate, lysozyme, tyloxapol, or a combination thereof.

65. The method of any of claims 60, 62, 63, or 64, wherein the composition is bioadhesive.

66. A method of treating a subject in need comprising administering to the subject an effective amount of a composition comprising:
(a) particles of a triamcinolone or a salt thereof, wherein the triamcinolone particles have an effective average particle size of less than about 2000 nm; and
(b) at least one surface stabilizer.

67. The method of claim 66, wherein the triamcinolone is selected from the group consisting of triamcinolone, triamcinolone acetonide, triamcinolone diacetate, triamcinolone hexacetonide, and triamcinolone benetonide.

68. The method of claim 67, wherein the triamcinolone is triamcinolone acetonide.

69. The method of claim 66, wherein the triamcinolone or a salt thereof is selected from the group consisting of a crystalline phase, an amorphous phase, a semi-crystalline phase, a semi-amorphous phase, and mixtures thereof.

70. The method of claim 66, wherein the effective average particle size of the triamcinolone particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

71. The method of claim 66, wherein the composition is formulated for administration selected from the group consisting of oral, pulmonary, rectal, opthalmic, colonic, parenteral, intracisternal, intravaginal, intraperitoneal, local, buccal, nasal, and topical administration.

72. The method of claim 66, wherein the composition further comprises one or more pharmaceutically acceptable excipients, carriers, or a combination thereof.

73. The method of claim 66, wherein the triamcinolone or salt thereof is present in an amount selected from the group consisting of from about 99.5% to about 0.001%, from about 95% to about 0.1%, and from about 90% to about 0.5%, by weight, based on the total combined dry weight of the triamcinolone or salt thereof and at least one surface stabilizer, not including other excipients.

74. The method of claim 66, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the triamcinolone or a salt thereof and at least one surface stabilizer, not including other excipients.

75. The method of claim 66, utilizing at least two surface stabilizers.

76. The method of claim 66, wherein at least one surface stabilizer is selected from the group consisting of an anionic surface stabilizer, a cationic surface stabilizer, a zwitterionic surface stabilizer, and an ionic surface stabilizer.

77. The method of claim 76, wherein at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

78. The method of claim 76, wherein at least one cationic surface stabilizer is selected from the group consisting of a polymer, a biopolymer, a polysaccharide, a cellulosic, an alginate, a nonpolymeric compound, and a phospholipid.

79. The method of claim 76, wherein the surface stabilizer is selected from the group consisting of benzalkonium chloride, polymethylmethacrylate trimethylammonium bromide, polyvinylpyrrolidone-2-dimethylaminoethyl methacrylate dimethyl sulfate, hexadecyltrimethyl ammonium bromide, cationic lipids, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₆dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl(ethenoxy)₄ ammonium bromide, N-alkyl (C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUA™, alkyl pyridinium salts; amines, amine salts, amine oxides, imide azolinium salts, protonated quaternary acrylamides, methylated quaternary polymers, and cationic guar.

80. The method of claim 66, comprising as a surface stabilizer a random copolymer of vinyl pyrrolidone and vinyl acetate, sodium lauryl sulfate, lysozyme, tyloxapol, or a combination thereof.

81. The method of any of claims 76, 78, 79, or 80, wherein the composition is bioadhesive.

82. The method of claim 66, additionally comprising administering one or more non-triamcinolone active agents.

83. The method of claim 82, wherein said additional one or more non-triamcinolone active agents are selected from the group consisting of nutraceuticals, amino acids, proteins, peptides, nucleotides, anti-obesity drugs, central nervous system stimulants, carotenoids, corticosteroids, elastase inhibitors, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, alpha-adrenergic receptor blocking agents, beta-adrenoceptor blocking agents, blood products, blood substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, parathyroid biphosphonates, prostaglandins, radio-pharmaceuticals, sex hormones, anti-allergic agents, stimulants, anoretics, sympathomimetics, thyroid agents, vasodilators, and xanthines.

84. The method of claim 82, wherein said additional one or more non-triamcinolone active agents are selected from the group consisting of acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

85. The method of claim 82, further comprising administering at least one antihistamine, decongestant, bronchodilator, anti-fungal, anti-cancer agent, or immunosuppressant.

86. The method of claim 85, wherein the antihistamine is selected from the group consisting of fexofenadine, azelastine, hydoxyzine, diphenhydramine, loratadine, chlorpheniramine maleate, cyproheptadine, promethazine, phenylephrine tannate, acrivastine, and cetirizine.

87. The method of claim 85, wherein the decongestant is selected from the group consisting of pseudoephedrine, oxymetazoline, xylometazoline, naphazoline, naphazoline, and tetrahydrozoline.

88. The method of claim 85, wherein the bronchodilator is selected from the group consisting of short-acting beta2-agonists, long-acting beta2-agonists, anticholinergics, and theophyllines.

89. The method of claim 85, wherein the anti-fungal agent is selected from the group consisting of amphotericin B, nystatin, fluconazole, ketoconazole, terbinafine, itraconazole, imidazole, triazole, ciclopirox, clotrimazole, and miconazole.

90. The method of claim 66, wherein the Tₘₐₓ of the triamcinolone composition, when assayed in the plasma of a mammalian subject following administration, is less than the Tₘₐₓ for a non-nanoparticulate composition of the same triamcinolone, administered at the same dosage.

91. The method of claim 90, wherein the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by the non-nanoparticulate composition of the same triamcinolone, administered at the same dosage.

92. The method of claim 66, wherein the Cₘₐₓ of the triamcinolone composition, when assayed in the plasma of a mammalian subject following administration, is greater than the Cₘₐₓ for a non-nanoparticulate composition of the same triamcinolone, administered at the same dosage.

93. The method of claim 92, wherein the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate formulation of the same triamcinolone, administered at the same dosage.

94. The method of claim 66, wherein the AUC of the triamcinolone composition, when assayed in the plasma of a mammalian subject following administration, is greater than the AUC for a non-nanoparticulate composition of the same triamcinolone, administered at the same dosage.

95. The method of claim 94, wherein the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate composition of the same triamcinolone, administered at the same dosage.

96. The method of claim 66, wherein the triamcinolone composition does not produce significantly different absorption levels when administered under fed as compared to fasting conditions.

97. The method of claim 96, wherein the difference in absorption of the triamcinolone composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

98. The method of claim 66, wherein administration of the composition to a human in a fasted state is bioequivalent to administration of the composition to a human in a fed state.

99. The method of claim 98, wherein “bioequivalency” is established by a 90% Confidence Interval of between 0.80 and 1.25 for both Cₘₐₓ and AUC.

100. The method of claim 98, wherein “bioequivalency” is established by a 90% Confidence Interval of between 0.80 and 1.25 for AUC and a 90% Confidence Interval of between 0.70 to 1.43 for Cₘₐₓ.

101. The method of claim 66, wherein the subject is a human.

102. The method of claim 66, wherein the method is used to treat indications where glucocorticoids are typically used.

103. The method of claim 66, wherein the method is used to treat indications where steroidal anti-inflammatory agents are typically used.

104. The method of claim 66, wherein the method is used to treat indications selected from the group consisting of arthritis, skin disorders, blood disorders, kidney disorders, eye disorders, thyroid disorders, intestinal disorders, allergies, asthma, bronchial asthma, cancer, neoplastic diseases, tendinitis, allergic reactions, seasonal allergic rhinitis, perennial allergic rhinitis, oral inflammation, oral lesions, oral ulcers, bursitis, epicondylitis, keloids, endocrine disorders, herpes zoster ophthalmicus, hemolytic anemia, and acute rheumatic carditis.

105. The method of claim 104, wherein the skin disorder is selected from the group consisting of contact dermatitis, atopic dermatitis, psoriasis, eczema, and general dermatitis.

106. The method of claim 104, wherein the arthritic condition is selected from the group consisting of osteoarthritis, acute nonspecific osteoarthritis, posttraumatic osteoarthritis, and rheumatoid arthritis.

107. The method of claim 104, wherein the intestinal disorder is selected from the group consisting of ulcerative colitis, colitis, gastroenteritis, irritable bowel disorder, and Crohn's disease.

108. The method of claim 66, wherein the method is used to treat indications selected from the group consisting of asthma, seasonal allergic rhinitis, and perennial allergic rhinitis.
